(19) United States
(12) Patent Application Publication
(10) Pub. No.: US 2024/0330140 A1
(43) Pub. Date: Oct. 3, 2024

(54) CONFIGURABLE AND ADAPTABLE OBSERVATION INTERVALS AND REPORTING INTERVALS FOR A MULTI-SENSOR NETWORK ENVIRONMENT

(71) Applicant: Thomson Licensing, Cesson-Sevigne (FR)

(72) Inventors: Navneeth N. KANNAN, Doylestown, PA (US); David GOODWIN, Southampton, PA (US); Cesar A. MORENO, Port Saint Joe, FL (US); Jay CHAMBERS, Rogers, AR (US); William RYAN, Los Angeles, CA (US)

(21) Appl. No.: 18/128,469

(22) Filed: Mar. 30, 2023

(51) Int. Cl. G06F 11/30

(52) U.S. Cl. CPC G06F 11/3068; G06F 11/3058; G06F 11/3089

(57) ABSTRACT

A network device provides for configuring and adapting an observation interval and a reporting interval for a sensing device so as to conserve resources of the sensing device. The network device can store information associated with an observation interval and a reporting interval and modify the associated timing interval based on user input, an indication of life of a power source of the sensing device, data associated with a client user, for example, data received from a sensing device associated with a client user, or both. The network device can adapt the observation interval, the reporting interval, or both for any one or more sensing devices so as to conserve resources of the sensing device, for example, to conserve life of a power source of the sensing device.

### BACKGROUND

In general, a network environment can include various network devices including multiple devices that sense various aspects of the environment associated with a user. The collection of data from network devices in a network environment can become unmanageable and require consumption of valuable resources, such as battery life of a network device. There is a need to provide collection of data from various network devices while conserving valuable resources.

### SUMMARY

Generally, there are many network devices in the market that operate or behave as point solutions for specific monitoring of aspects associated with a user. Accumulating and/or analyzing the data or information from these various solutions or technologies can consume valuable resources including processing time, power consumption and network bandwidth. For network devices with a built-in power source (for example, a battery), such obtaining and reporting of data is especially sensitive and can cause the observing and reporting of data to increase costs to a point that the network device is no longer optimal or useful for a particular network environment. Especially in a caregiving network environment where a user is associated with a plurality of sensing device, the collection and reporting of the data can become expansive to the point that the sensing device requires undue or excessive maintenance to maintain the power supply. For example, when the observation interval and the reporting interval are too often, a battery-powered sensing device can require maintenance to charge or replace a battery power source which can increases the ongoing costs of operation of the sensing device especially given that a user may need not be capable of performing such maintenance and will require the assistance of a third-party.

According to one or more aspects of the present disclosure, an observation interval and/or a reporting interval associated with a sensing device can be configurable and adaptable so as to conserve valuable resources. An observation interval is an amount of time between each sensing of data by the sensing device. A reporting interval is an amount of time between transmission of data from the sensing device to another network device. During an observation interval, a network device receives sensor data from various sensing devices within, part of, or coupled to the network device with the sensor data being reported to another network device and/or a network resource at a reporting interval. According to one or more novel aspects of the present disclosure, the observation interval and the reporting interval can be configurated based on one or more resource factors, such as any of the type of sensor, type of sensor data, type of event, an expected or predicted life of the power source, an activity status, any other factor or any combination thereof. The observation interval and reporting interval can be separately and distinctly configured with each also being adaptable based on historical information associated with the one or more resource factors. The observation interval and the reporting interval may be configurable and adaptable for each sensing device. One or more types of events can supersede the observation interval and the reporting interval such that data associated with such an event can be sensed by the sensing device, reported by the sensing device to the network device, or both in real-time or some other frequencies different from either of the observation interval or the reporting interval. In this way, the life of the power source is maximized without unduly impacting the utilization of the data by the network device.

An aspect of the present disclosure provides a network device for providing one or more observation intervals and one or more reporting intervals associated with one or more sensing device in a network environment. The network device comprises a memory storing one or more computer-readable instructions and a processor configured to execute the one or more computer-readable instructions to configure one or more interval formats associated with the one or more sensing devices, wherein the one or more interval formats comprise the one or more observation intervals and the one or more reporting intervals, provide the one or more interval formats to the one or more sensing devices, and receive data from the one or more sensing devices based on the one or more interval formats.

In an aspect of the present disclosure, the processor is further configured to execute the one or more instructions to adapt at least one of the one or more interval formats based on information associated with a life of a power source of the at least one of the one or more interval formats.

In an aspect of the present disclosure, the one or more interval formats comprise a sensing device identifier, a resource factor, an interval type, and a timing interval associated with the one or more sensing device identifiers.

In an aspect of the present disclosure, the resource factor comprises any of a type of sensor, a type of sensor data, a type of event, an expected or predicted life of the power source, an activity status, any other factor or any combination thereof.

In an aspect of the present disclosure, the timing interval indicates an amount of time between obtaining data, reporting data, or both.

In an aspect of the present disclosure, the processor is further configured to execute the one or more instructions to compare a threshold associated with the resource factor to the data from at least one of the one or more sensing devices, and instruct the at least one sensing device to send additional data based on an adapted timing format.

In an aspect of the present disclosure, the processor is further configured to execute the one or more instructions to transmit the data to a network resource, receive one or more adapted interval formats from the network resource based on the data, and transmit the one or more adapted interval formats to at least one of the one or more sensing devices.

An aspect of the present disclosure provides a method for providing by a network device one or more observation intervals and one or more reporting intervals for one or more sensing device in a network environment. The method comprises configuring one or more interval formats associated with the one or more sensing devices, wherein the one or more interval formats comprise the one or more observation intervals and the one or more reporting intervals, providing the one or more interval formats to the one or more sensing devices, and receiving data from the one or more sensing devices based on the one or more interval formats.

In an aspect of the present disclosure, the method further comprises providing the one or more interval formats to the one or more sensing devices, and receiving data from the one or more sensing devices based on the one or more interval formats.

In an aspect of the present disclosure, the method such that the one or more interval formats comprise a sensing device identifier, a resource factor, an interval type, and a timing interval associated with the one or more sensing device identifiers.

In an aspect of the present disclosure, the method such that the one or more interval formats comprise a sensing device identifier, a resource factor, an interval type, and a timing interval associated with the one or more sensing device identifiers.

In an aspect of the present disclosure, the method such that the resource factor comprises any of a type of sensor, a type of sensor data, a type of event, an expected or predicted life of the power source, an activity status, any other factor or any combination thereof.

In an aspect of the present disclosure, the method such that the timing interval indicates an amount of time between obtaining data, reporting data, or both.

In an aspect of the present disclosure, the method further comprises comparing a threshold associated with the resource factor to the data from at least one of the one or more sensing devices, and instructing the at least one sensing device to send additional data based on an adapted timing format.

An aspect of the present disclosure provides a non-transitory computer-readable medium of a network device storing one or more instructions for providing one or more observation intervals and one or more reporting intervals associated with one or more sensing device in a network environment. The one or more instructions when executed by a processor of the network device, cause the network device to perform one or more operations including the steps of the methods described above.

Thus, according to various aspects of the present disclosure described herein, it is possible to provide an observation interval and a reporting interval that are independently configurable and adaptable based on sensor data received from one or more sensing devices of a network environment associated with a user. The novel solution(s) provide a network device that determines an observation interval and a reporting interval based on one or more factors so as to conserve resources while providing an enhanced user experience.

### BRIEF DESCRIPTION OF DRAWINGS

In the drawings, like reference numbers generally indicate identical, functionally similar, and/or structurally similar elements.

FIG. 1 is a schematic diagram of network devices in a network environment, according to one or more aspects of the present disclosure;

FIG. 2 is a more detailed block diagram illustrating various components of a network device, according to one or more aspects of the present disclosure;

FIG. 3 is an exemplary network device of a network environment, according to one or more aspects of the present disclosure;

FIG. 4 is an illustration of an example notification to a client user based on an observation interval a reporting interval, according to one or more aspects of the present disclosure;

FIGS. 5A and 5B are exemplary aspects of a profile configuration for a multi-modal portal system, according to one or more aspects of the present disclosure;

FIG. 6 is an illustration of an example configurable and adaptable interval format, according to one or more aspects of the present disclosure;

FIG. 7 is an illustration of an example configuration for one or more observation intervals and one or more reporting intervals, according to one or more aspects of the present disclosure; and

FIG. 8 is a flow chart illustrating a method of a network device for providing one or more observation intervals and one or more reporting intervals associated with one or more sensing devices in a network environment, according to one or more aspects of the present disclosure.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness. Those of ordinary skill in the art will recognize that various changes and modifications of the examples described herein can be made without departing from the spirit and scope of the present disclosure.

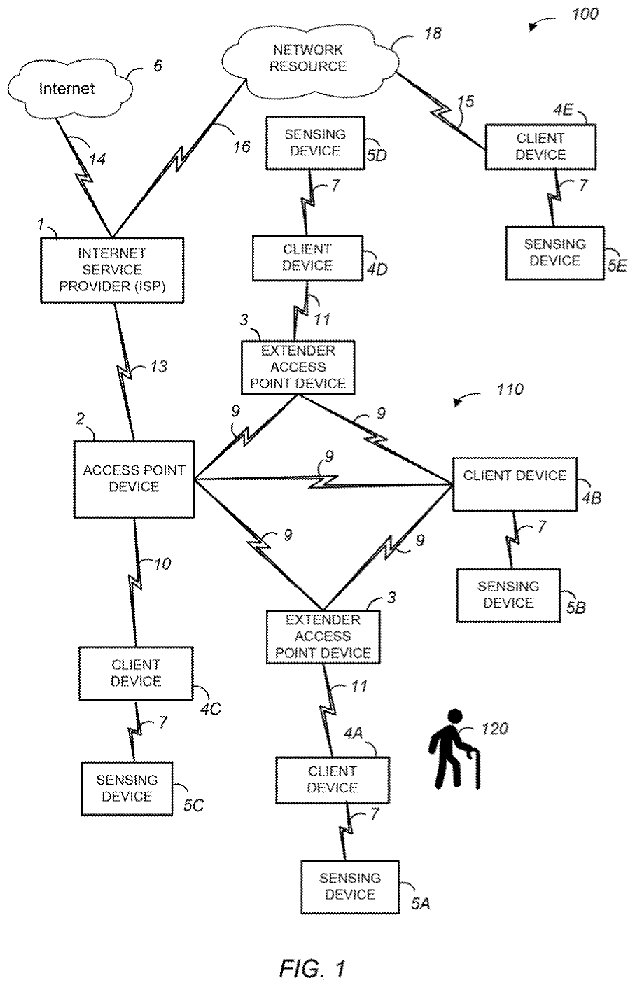

FIG. 1 is a schematic diagram of network devices in a network environment 100, according to one or more aspects of the present disclosure. For example, a user environment 110 associated with a client user to provide for aggregation of data from one or more network devices can be within or part of a network environment 100. An example network environment 100 can be related to a caregiving network for a user (a patient) such that one or more aspects associated with the user can be aggregated and/or monitored from one or more network devices capable of sensing the one or more aspects associated with a client user 120 of the user environment 110, such as any of biometric data, environmental data, any other data, or any combination thereof, at one or more observation intervals, one or more reporting intervals or both. Access to the aggregated and/or monitored data can be controlled based on one or more profile configurations as discussed with reference to FIGS. 5A-5B.

It should be appreciated that various example embodiments of inventive concepts disclosed herein are not limited to specific numbers or combinations of devices, and there may be one or multiple of some of the aforementioned electronic apparatuses in the network environment, which may itself consist of multiple communication networks and various known or future developed wireless connectivity technologies, protocols, devices, and the like.

The network environment 100 can include a user environment 110 that includes a network comprising an access point device 2 connected to a network resource such as any of the Internet 6, a network resource 18, any other cloud storage/repository, or any combination thereof via an Internet Service Provider (ISP) 1 and also connected to different wireless devices or network devices such as one or more wireless extender access point devices 3, one or more client devices 4A-4E (collectively referred to as client device(s) 4), and one or more sensing devices 5A-5E (collectively referred to as sensing device(s) 5). The network environment 100 shown in FIG. 1 includes wireless network devices (for example, extender access point devices 3 and client devices 4) that may be connected in one or more wireless networks (for example, private, guest, iControl, backhaul network, or Internet of things (IoT) network) within the network environment 100. Additionally, there could be some overlap between wireless devices (for example, extender access point devices 3 and client devices 4) in the different networks. That is, one or more network or wireless devices could be located in more than one network. For example, the extender access point devices 3 could be located both in a private network for providing content and information to a client device 4 and also included in a backhaul network or an iControl network.

The network environment 100 can include an ISP 1, for example, a content provider or any computer for connecting the access point device 2 to a network resource 18 and Internet 6. For example, Internet 6 can be a cloud-based service that provides access to a cloud-based repository accessible via ISP 1 where the cloud-based repository comprises information associated with or an access requested by any one or more network devices of the network environment 100. The network resource 18 can comprise a network device and can provide monitoring, aggregation and/or controlling of data associated with a client user 120 in the user environment 120, such as data collected by one or more sensing devices 5. The network resource 18 can comprise a user interface that allows for the configuration of one or more intervals associated with one or more sensing device 5, such as an observation interval, a reporting interval or both. In one or more embodiments, the network device 18 can communicate with any one or more external repositories of Internet 6 via ISP 1 or internal repositories. In one or more embodiments, any of the sensing devices 5 can be directly or indirectly coupled to the network resource 18. The connection 14 between the Internet 6 and the ISP 1, the connection 16 between the network resource 18 and the ISP 1, the connection 15 between the network resource 18 and the client device 5E, and the connection 13 between the ISP 1 and the access point device 2 can be implemented using a wide area network (WAN), a virtual private network (VPN), metropolitan area networks (MANs), system area networks (SANs), a data over cable service interface specification (DOCSIS) network, a fiber optics network (e.g., FTTH (fiber to the home) or FTTX (fiber to the x), or hybrid fiber-coaxial (HFC)), a digital subscriber line (DSL), a public switched data network (PSDN), a global Telex network, or a 2G, 3G, 4G, 5G, or 6G network, for example.

Any of the connections 13, 14, 15, 16, or any combination thereof (collectively referred to as network connections or connections) can further include as some portion thereof a broadband mobile phone network connection, an optical network connection, or other similar connections. For example, any of the network connections can also be implemented using a fixed wireless connection that operates in accordance with, but is not limited to, 3rd Generation Partnership Project (3GPP) Long Term Evolution (LTE), 5G, or 6G protocols. It is also contemplated by the present disclosure that any of the network connections are capable of providing connections between a network device and a WAN, a LAN, a VPN, MANs, PANs, WLANs, SANs, a DOCSIS network, a fiber optics network (e.g., FTTH, FTTX, or HFC), a PSDN, a global Telex network, or a 2G, 3G, 4G, 5G or 6G network, for example.

The access point device 2 can be, for example, an access point and/or a hardware electronic device that may be a combination modem and gateway that combines the functions of a modem, an access point (AP), and/or a router for providing content received from the ISP 1 to one or more network devices (e.g., wireless extender access point devices 3 and client devices 4) in the network environment 100, or any combination thereof. It is also contemplated by the present disclosure that the access point device 2 can include the function of, but is not limited to, a universal plug and play (UPnP) simple network management protocol (SNMP), an Internet Protocol/Quadrature Amplitude Modulator (IP/QAM) set-top box (STB) or smart media device (SMD) that is capable of decoding audio/video content, and playing over-the-top (OTT) or multiple system operator (MSO) provided content. The access point device 2 may also be referred to as a residential gateway, a home network gateway, or a wireless access point (AP).

The connection 9 between the access point device 2 and the wireless extender access point devices 3, and client device 4B can be implemented using a wireless connection in accordance with any IEEE 802.11 Wi-Fi protocols, Bluetooth protocols, Bluetooth Low Energy (BLE), or other short range protocols that operate in accordance with a wireless technology standard for exchanging data over short distances using any licensed or unlicensed band such as the citizens broadband radio service (CBRS) band, 2.4 GHz bands, 5 GHz bands, 6 GHz bands, or 60 GHz bands. Additionally, the connection 9 can be implemented using a wireless connection that operates in accordance with, but is not limited to, RF4CE protocol, ZigBee protocol, Z-Wave protocol, or IEEE 802.15.4 protocol. It is also contemplated by the present disclosure that the connection 9 can include connections to a media over coax (MoCA) network. One or more of the connections 9 can also be a wired Ethernet connection. Any one or more of connections 9 can carry information on any of one or more channels that are available for use.

The extender access point devices 3 can be, for example, wireless hardware electronic devices such as access points (APs), extenders, repeaters, etc. used to extend the wireless network by receiving the signals transmitted by the access point device 2 and rebroadcasting the signals to, for example, client devices 4, which may be out of range of the access point device 2. The extender access point devices 3 can also receive signals from the client devices 4 and rebroadcast the signals to the access point device 2, or other client devices 4.

The connection 11 between the extender access point devices 3 and the client devices 4A and 4D are implemented through a wireless connection that operates in accordance with any IEEE 802.11 Wi-Fi protocols, Bluetooth protocols, BLE, or other short range protocols that operate in accordance with a wireless technology standard for exchanging data over short distances using any licensed or unlicensed band such as the CBRS band, 2.4 GHz bands, 5 GHz bands, 6 GHz bands, or 60 GHz bands. Additionally, the connection 11 can be implemented using a wireless connection that operates in accordance with, but is not limited to, RF4CE protocol, ZigBee protocol, Z-Wave protocol, or IEEE 802.15.4 protocol. Also, one or more of the connections 11 can be a wired Ethernet connection. Any one or more connections 11 can carry information on any one or more channels that are available for use.

The client devices 4 can be, for example, hand-held computing devices, personal computers, electronic tablets, mobile phones, smart phones, smart speakers, Internet-of-Things (IoT) devices, iControl devices, portable music players with smart capabilities capable of connecting to the Internet, cellular networks, and interconnecting with other devices via Wi-Fi and Bluetooth, or other wireless hand-held consumer electronic devices capable of executing and displaying content received through the access point device 2. Additionally, the client devices 4 can be a television (TV), an IP/QAM set-top box (STB) or a streaming media decoder (SMD) that is capable of decoding audio/video content, and playing over OTT or MSO provided content received through the access point device 2. Further, a client device 4 can be a network device that requires configuration by the access point device 2. A client device 4 can be configured the same as or similar to a network resource 18 so as to include a user interface that allows for the configuration of one or more intervals. In one or more embodiments, the client devices 4 can comprise any network device associated with a client user 120 for interacting with any type of one or more sensing devices 5. For example, the client device 4 can interact with a plurality of sensing devices 5 where each sensing device 5 senses one or more aspects associated with a client user 120 or an environment 110. In one or more embodiments, one or more sensing devices 5 are included within or local to (built-in) the client device 4.

One or more sensing devices 5 can connect to one or more client devices 4, for example, via a connection 7. Connection 7 can utilize any one or more protocols discussed above with respect to connection 9. Any of the one or more sensing devices 5 can comprise or be coupled to an optical instrument (such as a camera, an image capture device, any other visual user interface device, any device for capturing an image, a video, a multi-media video, or any other type of data, or a combination thereof), a biometric sensor, a biometric tracker, ambient temperature sensor, a light sensor, a humidity sensor, a motion detector (such as, an infrared motion sensor or Wi-Fi motion sensor), a facial recognition system, a medical diagnostic sensor (such as, a pulse oximeter or any other oxygen saturation sensing system, a sound detection sensor, a blood pressure monitor, a temperature sensor, a glucose monitor, etc.), a voice recognition system, a microphone (such as, a far field voice (FFV) microphone) or other audio and/or sound capture system, any other sensing device, or a combination thereof.

The connection 10 between the access point device 2 and the client device 4 is implemented through a wireless connection that operates in accordance with, but is not limited to, any IEEE 802.11 protocols. Additionally, the connection 10 between the access point device 2 and the client device 4C can also be implemented through a WAN, a LAN, a VPN, MANs, PANs, WLANs, SANs, a DOCSIS network, a fiber optics network (e.g., FTTH, FTTX, or HFC), a PSDN, a global Telex network, or a 2G, 3G, 4G, 5G or 6G network, for example.

The connection 10 can also be implemented using a wireless connection in accordance with Bluetooth protocols, BLE, or other short range protocols that operate in accordance with a wireless technology standard for exchanging data over short distances using any licensed or unlicensed band such as the CBRS band, 2.4 GHz bands, 5 GHz bands, 6 GHz bands or 60 GHz bands. One or more of the connections 10 can also be a wired Ethernet connection. In one or more embodiments, any one or more client devices 4 utilize a protocol different than that of the access point device 2.

It is contemplated by the present disclosure that the network devices of the network environment 100, such as the network resource 18, the access point device 2, the extender access point devices 3, and/or the client devices 4, include electronic components or electronic computing devices operable to receive, transmit, process, store, and/or manage data and information associated with the network environment 100, which encompasses any suitable processing device adapted to perform computing tasks consistent with the execution of computer-readable instructions stored in a memory or a computer-readable recording medium (for example, a non-transitory computer-readable medium).

Further, any, all, or some of the computing components in the network resource 18, access point device 2, the extender access point devices 3, and the client devices 4 may be adapted to execute any operating system, including Linux, UNIX, Windows, MacOS, DOS, and ChromOS as well as virtual machines adapted to virtualize execution of a particular operating system, including customized and proprietary operating systems. The network resource 18, the access point device 2, the extender access point devices 3, and the client devices 4 are further equipped with components to facilitate communication with other computing devices or network devices over the one or more network connections to local and wide area networks, wireless and wired networks, public and private networks, and any other communication network enabling communication in the network environment 100.

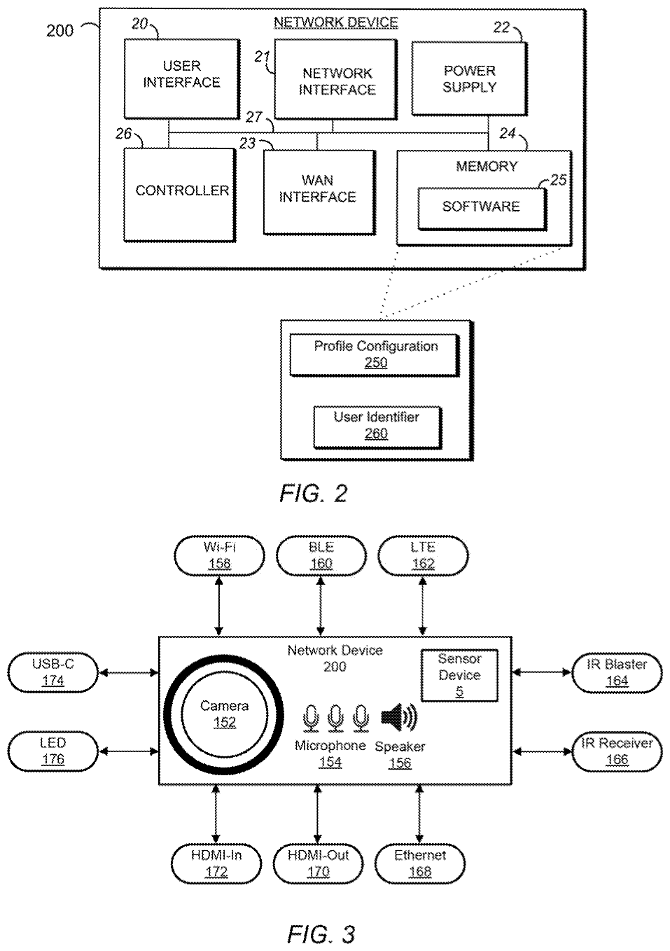

FIG. 2 is a more detailed block diagram illustrating various components of an exemplary network device 200, such as a network resource 18, an access point device 2, an extender access point device 3, a client device 4, any other network device, or any combination thereof implemented in the network environment 100 of FIG. 1, according to one or more aspects of the present disclosure.

The network device 200 can be, for example, a computer, a server, any other computer device with smart capabilities capable of connecting to the Internet, cellular networks, and interconnecting with other network devices via Wi-Fi and Bluetooth, or other wireless hand-held consumer electronic device capable of providing management and control of user data, for example, a network resource 18, according to one or more aspects of the present disclosure. The network device 200 includes one or more internal components, such as a user interface 20, a network interface 21, a power supply 22, a controller 26, a WAN interface 23, a memory 34, and a bus 27 interconnecting the one or more elements.

The power supply 22 supplies power to the one or more internal components of the network device 200 through the internal bus 27. The power supply 22 can be a self-contained power source such as a battery pack with an interface to be powered through an electrical charger connected to an outlet (e.g., either directly or by way of another device). The power supply 22 can also include a rechargeable battery that can be detached allowing for replacement such as a nickel-cadmium (NiCd), nickel metal hydride (NiMH), a lithium-ion (Li-ion), or a lithium Polymer (Li-pol) battery.

The user interface 20 includes, but is not limited to, push buttons, a keyboard, a keypad, a liquid crystal display (LCD), a thin film transistor (TFT), a light-emitting diode (LED), a high definition (HD) or other similar display device including a display device having touch screen capabilities so as to allow interaction between a user and the network device 200, for example, for a user to enter any one or more profile configurations 250, a user identifier 260, any other information associated with a user or network device, or a combination thereof that are stored in memory 34. The network interface 20 can include, but is not limited to, various network cards, interfaces, and circuitry implemented in software and/or hardware to enable communications with and/or between the network resource 18, the access point device 2, an extender access point device 3, and/or a client device 4 using any one or more of the communication protocols in accordance with any one or more connections (for example, as described with reference to FIG. 1). In one or more embodiments, the user interface 20 enables communications with a sensing device 5, directly or indirectly so as to configure an observation interval, a reporting interval, or both.

The memory 24 includes a single memory or one or more memories or memory locations that include, but are not limited to, a random access memory (RAM), a dynamic random access memory (DRAM) a memory buffer, a hard drive, a database, an erasable programmable read only memory (EPROM), an electrically erasable programmable read only memory (EEPROM), a read only memory (ROM), a flash memory, logic blocks of a field programmable gate array (FPGA), an optical storage system, a hard disk or any other various layers of memory hierarchy. The memory 24 can be used to store any type of instructions, software, or algorithms including software 25, for example, a multi-modal portal application, for controlling the general function and operations of the network device 200 in accordance with one or more embodiments. In one or more embodiments, memory 24 can store any one or more profile configurations 250 associated with one or more user identifiers 260 so as to provide (aggregation, monitoring, and control of data, such as data received from one or more sensing devices 5. For example, controlling or establishing a visual interface connection between two network devices, such as between a client device 4E and a client device 4C or between a network device associated with a client user 120 and a network device associated with a trusted user (also referred to as a trusted user device). The one or more user identifiers 260 can comprise a unique identifier associated with one or more users, one or more network devices, or both. The one or more user identifiers 260 can be associated with one or more profile configurations 250 which include information associated with one or more profiles of one or more users, such as a client user 120. In one or more embodiments, the profile configuration 250 and/or the user identifier 260 is stored in any type of storage medium local to or remote from the network device 200.

The controller 26 controls the general operations of the network device 200 and includes, but is not limited to, a central processing unit (CPU), a hardware microprocessor, a hardware processor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software including the software 25 which can include a multi-modal portal application in accordance with one or more embodiments. Communication between the components (for example, 20-26) of the network device 200 may be established using an internal bus 27.

The network interface 21 can include various network cards, interfaces, and circuitry implemented in software and/or hardware to enable communications with any one or more other network devices, for example, any of a client device 4, ISP 1, any other network device (for example, as described with reference to FIG. 1), or a combination thereof. The communications can utilize a visual interface connection that allows for a visual interface between two users, for example, between a client use 120 and a user associated with a user profile of a profile configuration 250. For example, the network interface 21 can include multiple radios or sets of radios (for example, a 2.4 GHz radio, one or more 5 GHz radios, and/or a 6 GHz radio), which may also be referred to as wireless local area network (WLAN) interfaces. In one or more embodiments, one radio or set of radios (for example, 5 GHz and/or 6 GHz radio(s)) provides a backhaul connection between the wireless extender access point device 3 and the access point device 2, and optionally other wireless extender access point device(s) 3. In one or more embodiments, the network resource 18 is connected to or is part of the access point device 2 such that a backhaul connection is established between the network resource 18 and one or more wireless extender access point devices 3. Another radio or set of radios (for example, 2.4 GHz, 5 GHz, and/or 6 GHz radio(s)) provides a fronthaul connection between the extender access point device 3 and one or more client device(s) 4.

The wide area network (WAN) interface 23 may include various network cards, and circuitry implemented in software and/or hardware to enable communications between the access point device 2 and the ISP 1 using the wired and/or wireless protocols in accordance with connection 13 (for example, as described with reference to FIG. 1).

FIG. 3 illustrates a network device 200, according to one or more aspects of the present disclosure. The network device 200 can include an optical instrument or an image capture device (such as a camera 152 or any other device that can obtain one or more visuals of a client user), an audio input device (such as a microphone 154, a microphone array, a far field voice (FFV) solution, etc.), an audio output device (such as a speaker 156), a sensing device 5, and a network device 200. In one or more embodiments, any one or more components of the network device 200 can be included within or connected to any other network device, such as any of a network resource 18, a sensing device 5, a client device 4, an access point device 2, an extender access point device 3, any other device, or any combination thereof. The multi-sensor hub 150 can include any of one or more ports or receivers, for example, a Wi-Fi (such as a Wi-Fi5 (dual-band simultaneous (DBS)) port 158, a BLE port 160, an LTE port 162, an infrared (IR) blaster port 164, and IR receiver port (166), an Ethernet port 168, an HDMI-Out port 170, an HDMI-In port 172, an external power supply (such as a universal serial bus type-C (USB-C), an LED output 176, or any combination thereof. The sensing device 5 can include any one or more types of sensors such as any of a power sensor, a temperature sensor, a light sensor, a humidity sensor, a motion sensor, a biometric sensor (such as a blood pressure monitor, oxygen saturation meter, pulse meter, etc.), any other type of sensor, or any combination thereof. The network device 200 can include software for allowing a client user 120 or a user associated with one or more access parameters that permit configuring an observation interval and/or a reporting interval based on one or more resource factors.

The one or more profile configurations 250 associated with a user identifier 260 for one or more user, such as a client user 120, can comprise one or more parameters. For example, FIGS. 5A-5B illustrate one or more profile configurations 250 for a client user 120, according to one or more aspects of the present disclosure. As an example, the one or more profile configurations 250 can be associated with a healthcare services network and the client user 120 can be an aging-in-place person. As illustrated in FIG. 5A, the one or more parameters of a profile configuration 250 can comprise one or more user profiles 502, one or more profile descriptions 504, one or more access parameters 506, one or more device identifiers 508, one or more encrypted credentials 510, one or more pre-authorization accesses 512, any other parameters associated with a user and/or network device, or a combination thereof.

The one or more user profiles 502 are associated with one or more users and/or network devices and can include, but are not limited to, any of a primary contact, a caregiver, a healthcare professional, a coordinator, a personal service, any other type of user and/or network device, or any combination thereof. In one or more embodiments, any of the one or more user profiles 502 can be designated as a trusted user, such as a user with access to configuring an observation interval, a reporting interval or both. The one or more user profiles 502 can be associated with one or more profile descriptions 504 including, but not limited to, any of a family member, friend, and/or guardian, a personal staff member or nurse, a doctor, a care administrator, a general staff member, a trusted user, any other description, or a combination thereof as illustrated in FIG. 5B. The one or more user profiles 502 can be associated with one or more access parameters 506.

The one or more access parameters 506 can include the types of data associated with a client user 120 or a network device 200 associated with a corresponding user profile 502 is allowed to access, such as to view, configure, modify, store, manage etc. In one or more embodiments, the access parameters 506 can include any alphanumeric characters, a binary value, or any other value. For example, as illustrated, a “Yes” indicates access to the data while a “No” indicates that the data is not accessible by the corresponding user profile 502. In one or more embodiments, a binary “1” or “0” could be used. The one or more access parameters 506 can include, but are not limited to, any of a video call, an image data (such as from a camera), a diagnostic data (such as heart rate, blood pressure, oxygen level, weight, activity level, temperature, etc.), a sensor data, an activity data, a protected data, a pre-authorization data, any other type of data, or a combination thereof as illustrated in FIG. 5B.

The creating or setting up of a profile configuration 250 can begin with assignment of roles to individuals and/or network devices associated with a patient (such as client user 120). In one or more embodiments, the default setting for any one or more access parameters 506 is no access, for example, a “No”. Any one or more default settings could be used for any one or more of the access parameters 506. In one or more embodiments, the one or more user profiles 502 can be updated or modified dynamically. A user identifier 260 can also be associated with a device identifier 508 such that an encrypted credential 510, a per-authorization access 512, or both can be associated with a user profile 502, a device identifier 508, or both. An encrypted credential 510 can be utilized by the network resource 18 to provide authorization of a request from a user associated with a user profile 502. The pre-authorization access 512 can be associated with a user profile 502 such that a user associated with the user profile 502 is pre-authorized to access data associated with a client user 120, for example, pre-authorized to connect with a client user 120 via a visual interface connection.

While FIGS. 5A-5B illustrate one or more profile configurations 250 associated with a healthcare services network, the present disclosure contemplates that the one or more profile configurations 250 can be associated with any type of network. Additionally, the present disclosure contemplates that any one or more user profiles 502, one or more profile descriptions 504, one or more access parameters 506, one or more scheduling parameters, or any combination thereof can be added or deleted based on a particular network environment, including dynamically.

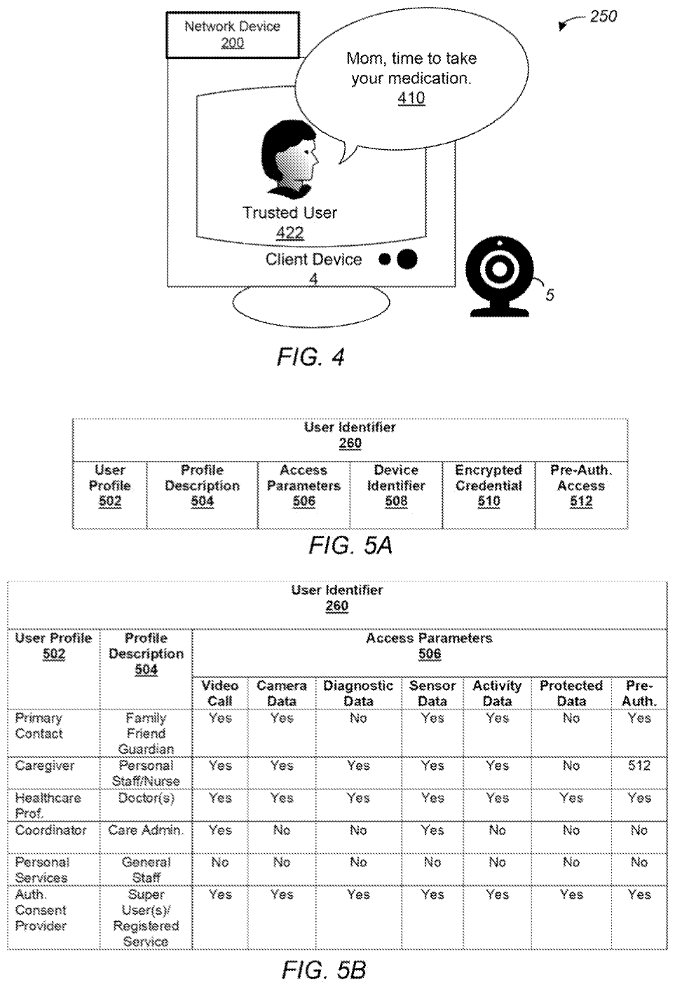

FIG. 4 is an illustration of an example notification to a client user based on an observation interval a reporting interval, according to one or more aspects of the present disclosure. For example, a network device 200 in a network 250 (for example, similar to or the same as user environment 110 of FIG. 1, can be connected to a client device 4, such as a display device that displays an audio/visual message. The network device 200 can be connected to a sensing device 5 that allows for the capture of user data, such as an image, an audio, or both. Based on an observation interval, the sensing device 5 can detect that a client user is present, for example, a client user 120, such as by sensing user data associated with an image and/or audio of the client user 120. The sensing device 5 can report the user data based on a reporting interval to the network device 200. For example, the network device 200 can determine based on a user profile 502 that the client user 120 has a scheduled dosage of medication. The network device 200 can update the observation interval the reporting interval associated with the sensing device 5 so as to report more frequently based on the scheduled dosage of medication. The network device 200 receives the user data from the sensing device 5 and determines that the client user 120 is at or about the client device 4. The network device sends a message 410 to the client device 4 so as to display the message 410 from a trusted user 422 associated with the user profile 502. After, the message 410 has been sent to the client device 4, the network device 200 can return the observation interval and/or the reporting interval for the sensing device 5 to the previous observation interval and/or the reporting interval, a default observation interval and/or a default reporting interval, or any other corresponding interval. In this way, the sensing device 5 does not observe and/or report user data more frequently than necessary to provide the client user 120 with a quality of service so as to conserve resources at the sensing device 5 and/or the network device 200. While FIG. 4 illustrates the network device 200, the client device 4, and the sensing device 5 as separate devices, the present disclosure contemplates that the network device 200 can comprise the client device 4, the sensing device 5, or both.

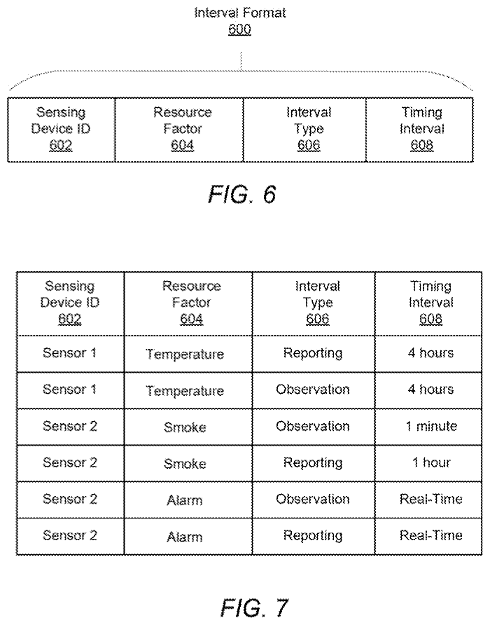

FIG. 6 is an illustration of an example configurable and adaptable interval format 600, according to one or more aspects of the present disclosure. An interval block 600 can comprise one or more elements, such as a sensing device identifier (ID) 602, a resource factor 604, an interval type 606, a timing interval 608, a reporting interval 610. The sensing device ID 602 can indicate a sensing device 5. The resource factor 604 can comprise any of the type of sensor, type of sensor data, type of event, an expected or predicted life of the power source, an activity status, any other factor or any combination thereof. The interval type 606 can indicate an observation interval, a reporting interval, or any other type of interval. The timing interval 608 can comprise an amount of time for the interval indicated by interval type 606, such as denoted by hours, minutes, second, and/or any other unit of time. The timing interval 608 indicates the amount of time that a network device 200, such as a sensing device 5, waits, pauses, or otherwise delays any of obtaining data (for example, from one or more sensors of the sensing device 5), referred to as an observation interval, transmitting the data obtained (for example, the data from one or more network devices 200, including, not limited to, one or more sensing devices 5, associated with a client user 120), referred to as a reporting interval.

FIG. 7 is an illustration of an example configuration for one or more observation intervals and one or more reporting intervals, according to one or more aspects of the present disclosure. For example, a sensing device ID 602 can indicate a Sensor 1 (for example, a temperature sensor) and a Sensor 2 (for example, a fire alarm). A resource factor 604 associated with the Sensor 1 indicates a type of sensor data of a temperature. A first resource factor 604 associated with the Sensor 2 indicates a type of sensor data of smoke while a second resource factor 604 associated with the Sensor 2 indicates a type of sensor data of an alarm. The interval type 606 and the timing interval 608 is associated with each sensing device ID 602. For example, the Sensor 1 has a reporting interval and observation interval every four hours associated with the resource factor 604 of temperature. The Sensor 2 has for a resource factor 604 of smoke an observation interval of 1 minute and a reporting interval of 1 hour such that the Sensor 2 obtains or receives smoke data every minute but only reports this smoke data to a network device 200 every hour. The Sensor 2 also has for a resource factor 604 of an alarm an observation interval and a report interval of real-time so that once the Sensor 2 detects smoke (based on an observation interval of 1 minute), the Sensor 2 will start obtaining and transmitting alarm data in real-time.

According to one or more aspects of the present disclosure, a sensing device 5 associated with a sensing device ID 602 can bundle environmental data obtained and event data determined by the sensing device 5. For example, the Sensor 2 can detect smoke or lack thereof as environmental data and also determine that smoke has been detected as an event data (such as an alarm). The Sensor 2 can transmit the environmental data (the smoke data) and the event data (the alarm due to detected smoke) as a bundled data set. This bundled data set can comprise data at the time of the event, prior to the event, and/or after the event. As another example, a glass break sound can be received as a sound input at a sound detection sensing device that triggers a microphone of the sound detection sensing device to obtain the sound input independent of an associated observation interval. The sound detection sensing device can determine that the sound input is a glass break and can trigger a glass break event that causes the sound detection sensing device to report data associated with the event to a network device 200 independent of an associated reporting interval. In this way, resources of a sensing device 5, for example, life of a power source of the sensing device 5, can be conserved while still providing data associated with one or more events in or about real-time.

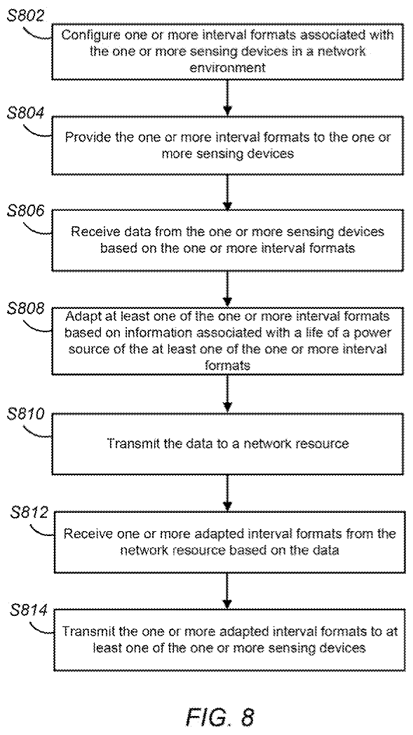

FIG. 8 is a flow chart illustrating a method of a network device 200 for providing one or more observation intervals and one or more reporting intervals associated with one or more sensing devices in a network environment, according to one or more aspects of the present disclosure.

The network device 200 may be programmed with one or more instructions that when executed by a processor or controller causes the network device 200 to for configuring an observation interval and a reporting interval. In FIG. 8, it is assumed that any one or more of the network devices 200 include their respective controllers and their respective software stored in their respective memories, as discussed above in connection with FIGS. 1-7, which when executed by their respective controllers perform the functions and operations in accordance with the example embodiments of the present disclosure (for example, including providing control of access to data associated with a client user 120 from one or more sensing devices 5).

The network device 200 comprises a controller 26 that executes one or more computer-readable instructions, stored on a memory 24, that when executed perform one or more of the operations of steps S802-S814. The network device 200 can comprise one or more software 25. While the steps S802-S814 are presented in a certain order, the present disclosure contemplates that any one or more steps can be performed simultaneously, substantially simultaneously, repeatedly, in any order or not at all (omitted).

At step S802, the network device 200 configures one or more interval formats associated with one or more sensing devices 5 in a network environment, such as network environment 100 and/or network 110 of FIG. 1. As an example, the one or more interval formats can comprise any one or more elements of an interval format 600, such as an interval type 606 associated with one or more timing intervals 608, such as an observation interval and a reporting interval. Configuring the one or more intervals formats, for example, the one or more interval formats as discussed with reference to FIG. 6, can be based on any one or more factors such as conservation of one or more resources. As an example, the one or more sensing devices 5 can be set to perform sampling of user sensor data based on one or more sampling intervals. To minimize power usage of a power source, such as a battery, an observation interval and/or a reporting interval of a timing interval can be configured or adapted for any one or more of the one or more sensing devices 5 in a network environment to collect and/or report samples or measurements based on dynamics of the network environment, such as biometric measurements, environmental measurements, any other measurements, user data, or any combination thereof. Generally, systems follow an exponential relationship that is usually dominated by a single pole or time constant. Such systems are oftentimes asymmetric, for example, a temperature of a room rises faster with direct heating that during cooling by natural convection. Similarly, humidity generally does not change rapidly unless, for example, a user takes a shower. The dynamic behavior can further change with a season of the year, the time of day or both. Optimization can be achieved by initially allowing one or more sensor devices 5 to passively observe the environment at a higher sampling rate than necessary (for example, a sampling interval of less than twenty seconds). A processor can then initiate a curve fitting algorithm (for example, exponential/polynomial) to determine an optimized sampling interval for the environment. Two dominant time constants (rising and falling) can also be derived for the environment. Combining this sampling interval determination with a measurement resolution (for example, 0.5 degrees Celsius, 1% relative humidity, etc.), a sampling interval associated with a sensing device 5 can dynamically be adjusted to conserve resources, such as to ensure minimum energy usage to prolong life of a power source (for example, a battery) so as to maximize time between charging requirements, when collecting user data. A timing interval can continuously, periodically, based on a threshold, etc. be adjusted, altered, or modified (collectively referred to as adapted) based on user data to provide an optimized timing interval for a particular sensing device 5. Any timing interval can be switched between a predetermined and/or default interval based on a trigger, such as a sensing of a person, anticipation of a rapid change in a user data, etc.

As an example, a default timing interval can be configured for each type of sensing device 5 so as to maximize one or more resources, for example, life of a power source, and conserve one or more resources at each sending device 5 and the monitoring system 180. For example, a temperature sensing device may only need a reporting interval and observation interval of a few times a day whereas a biometric sensing device may require an observation interval of many times a day with an intermittent (such as a triggered based on a comparison to a threshold) reporting interval.

As another example, a sound detection sensing device, as an example, can require a high power consumption compared to other sensing devices that do not require a digital signal processor. According to one or more aspects of the present disclosure, a sound detection sensing device can remove and/or reduce power to a digital signal processor so as to reduce overall power consumption. A sound input can be detected by a sound input element, such as a microphone, of the sound detection sensing device. The sound input can be compared to a sound threshold and only if the sound threshold is reached, exceeded or both, is power provided to the digital signal processor so as to detect a sound input for processing by the monitoring system 180. Further, each timing interval associated with each sensing device 5 can be dynamically and/or independently updated, modified or otherwise altered based on user data previously received by the monitoring system 180. For example, a motion detection sensing device can be associated with a first timing interval and can comprise a passive infrared detector device. Once the passive infrared detector device is triggered (for example, by movement of a person in a room), the motion detection sensing device can switch to a second sampling interval that is greater than the first sampling interval such that sampling occurs more often such that an activity or one or more other aspects associated with a user, a network, and/or a network environment can be detected as user sensor data. In this way, any one or more sensing devices can be associated with one or more timing intervals that are dynamically and/or independently modifiable and/or selectable so as to conserve resources (for example, improve life of a power source) while providing improved services to a user.

At step S804, the network device 200 provides the one or more interval formats to the one or more sensing devices 5. For example, the network device 200 can send to the one or more sensing devices 5 an instruction that comprises the one the one or more interval formats so as to cause the one or more sensing devices 5 to, for example, to implement an observation interval and reporting interval as indicated by the one or more interval formats.

At step S806, the network device 200 receives data, such as user data, from the one or more sensing devices 5 based on the one or more interval formats. The one or more interval formats can comprise any of a sensing device identifier, a resource factor (for example, that comprises any of a type of sensor, a type of sensor data, a type of event, an expected or predicted life of the power source, an activity status, any other factor or any combination thereof), an interval type, a timing interval associated with the one or more sensing device identifiers, or any combination thereof. The timing interval can indicate an amount of time the associated sensing device 5 waits between obtaining data, reporting data, or both. For example, the data can be received based on the observation interval and the reporting interval associated with the one or more interval formats. Any one or more sensing devices 5 can be associated with one or more interval formats. Any of the one or more sensing devices 5 can be associated with a different one or more of the one or more interval formats. For example, a first sensing device 5, for example, a motion detector, can be associated with a first interval format and a second interval format such that the first sensing device 5 observes and reports based on the first interval format until a sound input that is at and/or exceeds a sound threshold is detected and then observes and reports based on the second interval format.

At step S808, the network device 200 adapts at least one of the one or more interval formats based on information associated with a life of a power source of the at least one of the one or more interval formats. For example, the information can be or associated with the data received at step S806 which indicates a life of a power source of a sensing device. The adapting can comprise updating, altering, modifying, creating, deleting, and/or any other configuration of the at least one of the one or more interval formats. As an example, the network device 200 can compare a threshold associated with a resource factor to the data, for example, received at step S806, from at least one of the one or more sensing devices 5 and instruct the at least one sensing device 5 to send additional data based on an adapted timing format. The adapted timing format can comprise one or more adapted timing intervals, for example, an adapted observation interval, an adapted reporting interval, or both. In this way, any one or more elements of an interval format can be adapted to reflect data received from any of the one or more sensing devices 5.

At step S810, the network device 200 transmits the data to a network resource 18, for example, which is remote from the network device 200, such as a cloud resource. The network resource 18 can store, process, and/or otherwise analyze the data. For example, the network resource 18 can store the data in a repository, such as a database.

At step S812, the network device 200 can receive one or more adapted interval formats from the network resource 18 based on the data. For example, the network resource 18 can compare the data to a corresponding threshold and determine that an event has occurred or has not occurred such that one or more interval formats should be adapted.

At step S814, the network device 200 transmits the one or more adapted interval formats from step S812, to at least one of the one or more sensing devices 5 so as to cause the one the at least sensing device 5 to change, for example, a reporting interval, an observation interval, or both.

While the present disclosure discusses an aging-in-place environment, the present disclosure contemplates any other environment that requires any of remote monitoring, real-time analysis of data, dynamically adapted sampling (such as reporting interval and/or observation interval), or any combination thereof be provided within a secure and private network environment such that on-demand and/or pre-authorized can be provided as well as an audit trail or log can be maintained.

According to one or more example embodiments of inventive concepts disclosed herein, there are provided novel solutions for providing a configurable and an adaptable interval format for one or more sensing devices 5 in a network environment. The novel solutions according to example embodiments of inventive concepts disclosed herein provide features that enhance the installation and configuration of home/residential network gateway (GW) devices, wireless fidelity access points (Wi-Fi APs), Home Network Controller (HNC) devices, wireless routers, mesh networking nodes (e.g., Wi-Fi EasyMesh systems).

Each of the elements of the present invention may be configured by implementing dedicated hardware or a software program on a memory controlling a processor to perform the functions of any of the components or combinations thereof. Any of the components may be implemented as a CPU or other processor reading and executing a software program from a recording medium such as a hard disk or a semiconductor memory, for example. The processes disclosed above constitute examples of algorithms that can be affected by software, applications (apps, or mobile apps), or computer programs. The software, applications, computer programs or algorithms can be stored on a non-transitory computer-readable medium for instructing a computer, such as a processor in an electronic apparatus, to execute the methods or algorithms described herein and shown in the drawing figures. The software and computer programs, which can also be referred to as programs, applications, components, or code, include machine instructions for a programmable processor, and can be implemented in a high-level procedural language, an object-oriented programming language, a functional programming language, a logical programming language, or an assembly language or machine language.

The term “non-transitory computer-readable medium” refers to any computer program product, apparatus or device, such as a magnetic disk, optical disk, solid-state storage device (SSD), memory, and programmable logic devices (PLDs), used to provide machine instructions or data to a programmable data processor, including a computer-readable medium that receives machine instructions as a computer-readable signal. By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media.

The word “comprise” or a derivative thereof, when used in a claim, is used in a nonexclusive sense that is not intended to exclude the presence of other elements or steps in a claimed structure or method. As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. Use of the phrases “capable of,” “configured to,” or “operable to” in one or more embodiments refers to some apparatus, logic, hardware, and/or element designed in such a way to enable use thereof in a specified manner.

While the principles of the inventive concepts have been described above in connection with specific devices, apparatuses, systems, algorithms, programs and/or methods, it is to be clearly understood that this description is made only by way of example and not as limitation. The above description illustrates various example embodiments along with examples of how aspects of particular embodiments may be implemented and are presented to illustrate the flexibility and advantages of particular embodiments as defined by the following claims, and should not be deemed to be the only embodiments. One of ordinary skill in the art will appreciate that based on the above disclosure and the following claims, other arrangements, embodiments, implementations and equivalents may be employed without departing from the scope hereof as defined by the claims. It is contemplated that the implementation of the components and functions of the present disclosure can be done with any newly arising technology that may replace any of the above-implemented technologies. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

1. A network device for providing one or more observation intervals and one or more reporting intervals associated with one or more sensing devices in a network environment, an observation interval being an amount of time between each sensing of data by a sensing device of the one or more sensing devices, a reporting interval being an amount of time between transmission of data from the sensing device to another network device, comprising:
a memory storing one or more computer-readable instructions; and
a processor configured to execute the one or more computer-readable instructions to:
configure one or more interval formats associated with the one or more sensing devices based on conservation of one or more resources, wherein the one or more interval formats comprise the one or more observation intervals and the one or more reporting intervals, the observation intervals and the reporting intervals being separately and distinctly configured;
provide the one or more interval formats to the one or more sensing devices; and
receive data from the one or more sensing devices based on the one or more interval formats.

2. The network device of claim 1, wherein the processor is further configured to execute one or more instructions to:
adapt at least one of the one or more interval formats based on information associated with a life of a power source of the at least one of the one or more interval formats.

3. The network device of claim 1, wherein the one or more interval formats comprise a sensing device identifier, a resource factor, an interval type, and a timing interval associated with the one or more sensing device identifiers.

4. The network device of claim 3, wherein the resource factor comprises any of a type of sensor, a type of sensor data, a type of event, an expected or predicted life of the power source, an activity status, any other factor or any combination thereof.

5. The network device of claim 3, wherein the timing interval indicates an amount of time between obtaining data, reporting data, or both.

6. The network device of claim 3, wherein the processor is further configured to execute one or more instructions to:
compare a threshold associated with the resource factor to the data from at least one of the one or more sensing devices; and
instruct the at least one sensing device to send additional data based on an adapted timing format.

7. The network device of claim 1, wherein the processor is further configured to execute one or more instructions to:
transmit the data to a network resource;
receive one or more adapted interval formats from the network resource based on the data; and
transmit the one or more adapted interval formats to at least one of the one or more sensing devices.

8. A method for providing by a network device one or more observation intervals and one or more reporting intervals associated with one or more sensing devices in a network environment, an observation interval being an amount of time between each sensing of data by a sensing device of the one or more sensing devices, a reporting interval being an amount of time between transmission of data from the sensing device to another network device, the method comprising:
configuring one or more interval formats associated with the one or more sensing devices based on conservation of one or more resources, wherein the one or more interval formats comprise the one or more observation intervals and the one or more reporting intervals, the observation intervals and the reporting intervals being separately and distinctly configured;
providing the one or more interval formats to the one or more sensing devices; and
receiving data from the one or more sensing devices based on the one or more interval formats.

9. The method of claim 8, further comprising:
adapting at least one of the one or more interval formats based on information associated with a life of a power source of the at least one of the one or more interval formats.

10. The method of claim 8, wherein the one or more interval formats comprise a sensing device identifier, a resource factor, an interval type, and a timing interval associated with the one or more sensing device identifiers.

11. The method of claim 10, wherein the resource factor comprises any of a type of sensor, a type of sensor data, a type of event, an expected or predicted life of the power source, an activity status, any other factor or any combination thereof.

12. The method of claim 10, wherein the timing interval indicates an amount of time between obtaining data, reporting data, or both.

13. The method of claim 10, further comprising:
comparing a threshold associated with the resource factor to the data from at least one of the one or more sensing devices; and
instructing the at least one sensing device to send additional data based on an adapted timing format.

14. The method of claim 8, further comprising:
transmitting the data to a network resource;
receiving one or more adapted interval formats from the network resource based on the data; and
transmitting the one or more adapted interval formats to at least one of the one or more sensing devices.

15. A non-transitory computer-readable medium of a network device storing one or more instructions for providing one or more observation intervals and one or more reporting intervals associated with one or more sensing devices in a network environment, an observation interval being an amount of time between each sensing of data by a sensing device of the one or more sensing devices, a reporting interval being an amount of time between transmission of data from the sensing device to another network device, which when executed by a processor of the network device, cause the network device to perform one or more operations comprising:
configuring one or more interval formats associated with the one or more sensing devices based on conservation of one or more resources, wherein the one or more interval formats comprise the one or more observation intervals and the one or more reporting intervals, the observation intervals and the reporting intervals being separately and distinctly configured;
providing the one or more interval formats to the one or more sensing devices; and
receiving data from the one or more sensing devices based on the one or more interval formats.

16. The non-transitory computer-readable medium of claim 15, wherein the one or more instructions when executed by the processor further cause the network device to perform the one or more further operations comprising:
adapting at least one of the one or more interval formats based on information associated with a life of a power source of the at least one of the one or more interval formats.

17. The non-transitory computer-readable medium of claim 15, wherein the one or more interval formats comprise a sensing device identifier, a resource factor, an interval type, and a timing interval associated with the one or more sensing devices identifiers.

18. The non-transitory computer-readable medium of claim 17, wherein at least one of:
the resource factor comprises any of a type of sensor, a type of sensor data, a type of event, an expected or predicted life of the power source, an activity status, any other factor or any combination thereof; and
the timing interval indicates an amount of time between obtaining data, reporting data, or both.

19. The non-transitory computer-readable medium of claim 17, wherein the one or more instructions when executed by the processor further cause the network device to perform one or more further operations comprising:
comparing a threshold associated with the resource factor to the data from at least one of the one or more sensing devices; and
instructing the at least one sensing device to send additional data based on an adapted timing format.

20. The non-transitory computer-readable medium of claim 15, wherein the one or more instructions when executed by the processor further cause the network device to perform one or more further operations comprising:
transmitting the data to a network resource;
receiving one or more adapted interval formats from the network resource based on the data; and
transmitting the one or more adapted interval formats to at least one of the one or more sensing devices.
